(19) United States
(12) Patent Application Publication
(10) Pub. No.: US 2018/0265834 A1
(43) Pub. Date: Sep. 20, 2018

(54) DISPOSABLE CELL REMOVAL SYSTEM

(71) Applicant: Bend Research Inc., Bend, OR (US)

(72) Inventors: David Dixon Newbold, Bend, OR (US); Clinton Boyd Pepper, Bend, OR (US); David Andrew Hansen, Bend, OR (US)

(73) Assignee: Bend Research Inc., Bend, OR (US)

(21) Appl. No.: 15/532,959

(22) PCT Filed: Nov. 17, 2015
(86) PCT No.: PCT/IB2015/058869

§ 371 Date: Jun. 2, 2017

Related U.S. Application Data
(60) Provisional application No. 62/086,898, filed on Dec. 3, 2014.

(51) Int. Cl. C12M 1/00; B01L 3/00; C12M 3/00; C12M 1/26; B01D 61/18; B01D 63/02

(52) U.S. Cl. CPC C12M 47/02; B01L 3/502; C12M 23/42; C12M 23/28; C12M 33/14; C12M 33/04; B01D 2313/24; B01D 63/02; B01L 2300/0681; B01L 2400/0403; B01L 2400/0475; B01D 2313/20; B01D 61/18

(57) ABSTRACT

A cassette comprising: a first reservoir arranged to couple to a first actuator for imparting flow to a cell-containing solution in a first direction through a filter; a second reservoir arranged to couple to a second actuator for imparting flow to a cell-containing solution in a second direction through the filter; the filter being in fluid communication with the first and second reservoirs, and when connected to a filtration unit is in further fluid communication to a sample inlet, and a sample outlet; wherein, the cassette is disposable and housed by a connector being removeably adjoinable to the filtration unit, the filtration unit comprising the first and second actuators and one or more valves.

### FIELD

This disclosure relates to equipment and processes for the filtration of high solids content cell-containing solutions to produce cell-free solutions and/or samples for further analysis. Typically such equipment and processes being for integration into a bioreactor.

In particular, the disclosure relates to novel disposable filtration units and the filtration processes using such units.

### BACKGROUND

Cell culture in bioreactors has been target of considerable investment and innovation in the past decades. Examples of such bioreactors are described in U.S. Pat. No. 5,015,585A, U.S. Pat. No. 6,582,955B2, U.S. Pat. No. 6,616,912B2, WO2012154603A1, WO2013063128, and PCT/US2014/038614.

Once a cell solution is effectively generated, it is desirable to filter the solution to attain cell-free samples for further laboratory testing and analysis, and to obtain on-line process control and feedback to conditions within the bioreactor so that adjustments can be made. Examples of filtration systems that may be implemented are described in U.S. Pat. No. 7,172,696B1.

Such systems however have a number of disadvantages, such as long set-up time, turnaround time between batches, low scale-up opportunities, and difficulty in operating in an automatic/continuous manner.

Thus there still remains a need for an apparatus and process that effectively overcomes the problems of the prior art.

### SUMMARY

In a first aspect, the present disclosure relates to a cassette comprising: a first reservoir arranged to couple to a first actuator for imparting flow to a cell-containing solution in a first direction through a filter; a second reservoir arranged to couple to a second actuator for imparting flow to a cell-containing solution in a second direction through the filter; the filter being in fluid communication with the first and second reservoirs, and when connected to a filtration unit is in further fluid communication to a sample inlet, and a sample outlet; wherein, the cassette is disposable and housed by a connector being removeably adjoinable to the filtration unit, the filtration unit comprising the first and second actuators and one or more valves.

In a second aspect, the present disclosure relates to a filtration unit comprising the above mentioned cassette.

In a third aspect, the present disclosure relates to a process of filtering a cell comprising solution with the filtration unit and cassette.

### BRIEF DESCRIPTION OF THE DRAWINGS

FIG. 1 is a schematic drawing of a cassette according to one embodiment of the present disclosure.

FIG. 2 is a schematic drawing of a portion of a filtration unit according to one embodiment of the present disclosure.

FIG. 3 is a schematic drawing of a process diagram according to one embodiment of the present disclosure.

### DETAILED DESCRIPTION

The present disclosure relates to equipment and processes for filtering cell containing solutions to provide cell-free solutions. Various embodiments are disclosed herein. Although methods and materials similar or equivalent to those described herein can be used in the practice of the present technology, only certain suitable methods and materials are described herein. The following description is exemplary in nature and is not intended to limit the scope, applicability, or configuration of the disclosure(s) in any way. Various changes to the described embodiments may be made, such as in the function and arrangement of the elements described herein, without departing from the scope of the disclosure(s).

As used in this application and in the claims, the singular forms “a,” “an,” and “the” include the plural forms unless the context clearly dictates otherwise. Additionally, the term “includes” means “comprises.” Further, the term “coupled” generally means electrically, electromagnetically, and/or physically (e.g., mechanically or chemically) coupled or linked and does not exclude the presence of intermediate elements between the coupled or associated items absent specific contrary language.

The term “wetting component” as used herein means a component that comes in direct contact with a fluid (gas and/or liquid) and are typically selected from the group consisting of reservoirs, conduits, filters, and combinations thereof.

The term “substantially all wetting components” as used herein means all wetting components when filtering a solution (i.e. during the filtration step in a filtering process).

The term “cassette” as used herein means a cartridge capable of fitting and/or connecting to a filtration unit, typically the cartridge is a replaceable and/or disposable self-contained unit containing all components that, once fitted in the filtration unit, are automatically operated for filtering a solution.

The term “disposable” as used herein typically means that the element referred to may be disposed with and/or replaced after no more than 10 uses, preferably no more than 5 uses, preferably no more than 2 uses, most preferably no more than 1 use.

The term “uses or use”, as used herein means the procedure of filtering a same solution (e.g. formulation equivalent solution) before changing to another different solution (e.g. a formulation distinct solution).

Unless otherwise indicated, all numbers expressing quantities of ingredients, properties such as molecular weight, percentages, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods, as known by those of ordinary skill in the art. When directly and explicitly distinguishing embodiments from discussed prior art, the embodiment numbers are not approximates unless the word “about” is recited. Indeed, the dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” (i.e. every value in a practical range close to 40 mm).

Various embodiments will now be described to provide an overall understanding of the principles of the structure, function, manufacture, and use of dosage form articles and methods disclosed herein. One or more examples of these embodiments are illustrated in the accompanying figures. Those of ordinary skill in the art will immediately understand that features described or illustrated in connection with one example embodiment can be combined with the features of other example embodiments without generalization from the present disclosure

The Cassette

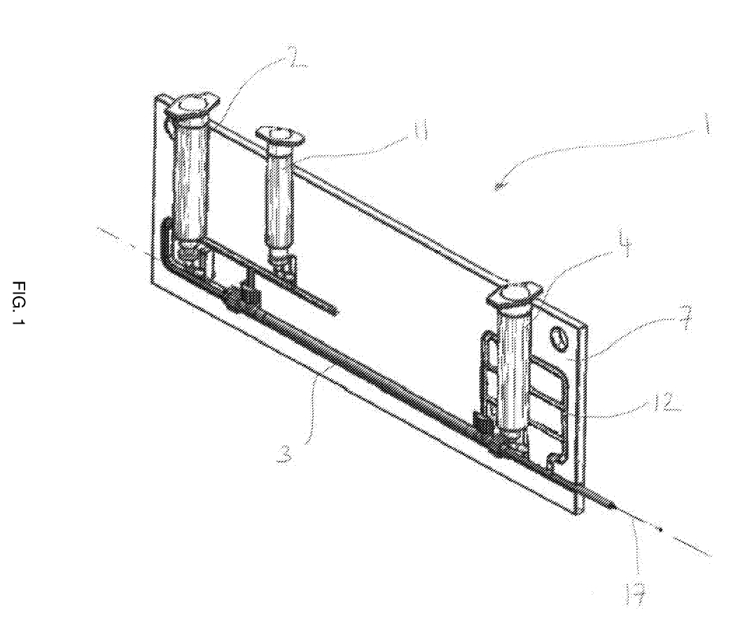

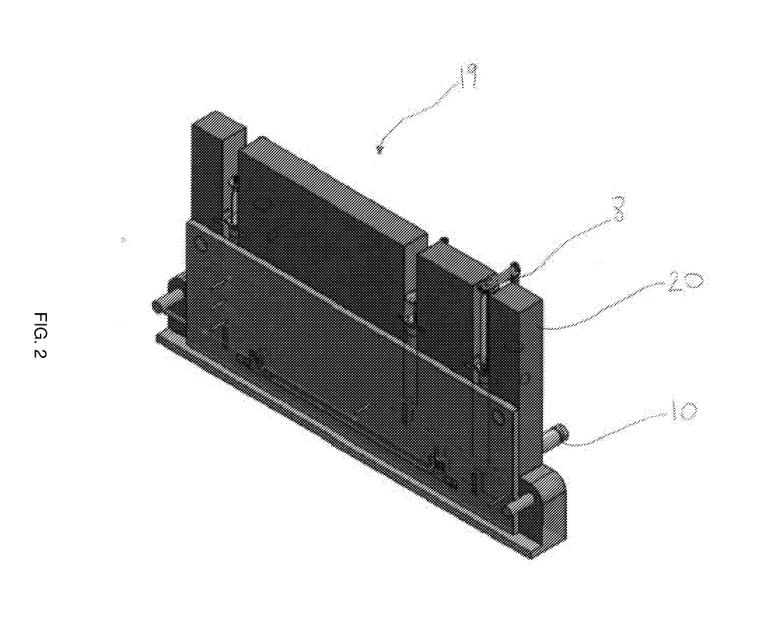

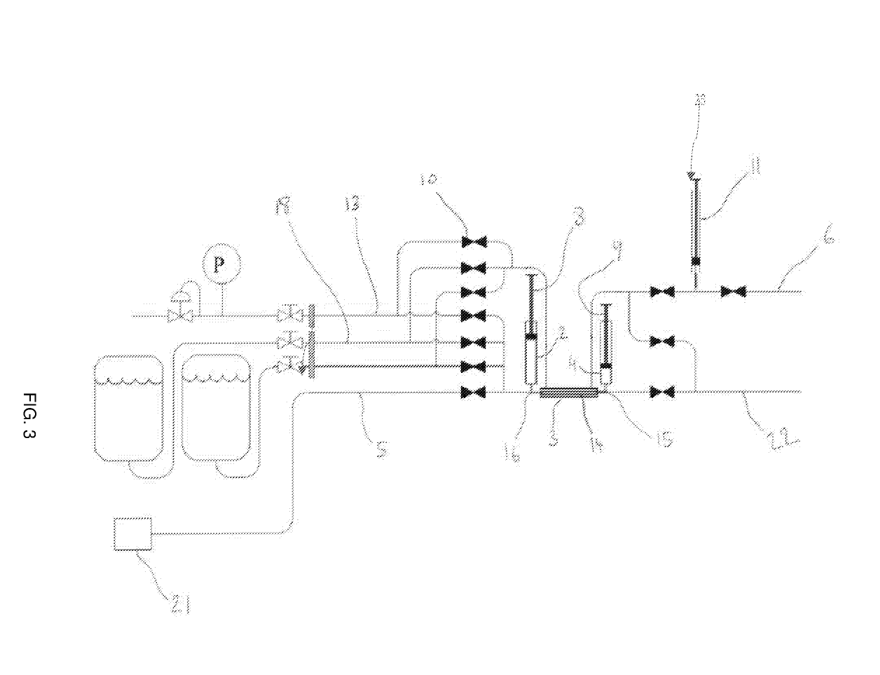

Referring to FIGS. 1 to 3, a cassette 1 according to the present disclosure comprises: a first reservoir 2 arranged to couple to a first actuator 8 for imparting flow to a cell-containing solution in a first direction through a filter 3; a second reservoir 4 arranged to couple to a second actuator 9 for imparting flow to a cell-containing solution in a second direction through the filter 3; the filter 3 being in fluid communication with the first and second reservoirs 2,4, and when connected to a filtration unit is in further fluid communication to a sample inlet 5, and a sample outlet 6 and typically a waste outlet 22; wherein, the cassette 1 is disposable and housed by a connector 7 being removeably adjoinable to the filtration unit, the filtration unit comprising the first and second actuators 8,9 and one or more valves 10, and optionally a third actuator 23 arranged to couple to the third reservoir 11 when the cassette 1 is connected to the filtration unit 19. Advantages of this arrangement include ease of replacement of wearing parts without needing to replace and dismount actuation systems, faster turnaround times, reduced risk of contamination or ineffective sampling by allowing ease of regular filter replacement.

In an embodiment, the first and second directions are different, preferably opposite directions. Advantages of this arrangement include effective sampling of the maximum amount of cell-free solution for a given amount of cell-comprising solution entered in the filtration loop and in an extremely compact and simple execution.

In an embodiment, the cassette 1 is free of moving parts, preferably selected from actuators, valves and combinations thereof. Advantages of this arrangement include limiting cost and complexity of the cassette for a truly disposable unit.

Preferably all components of the cassette 1 are made of a plastic material like polyolefins such as polyethylene (PE) and polypropylene (PP), polystyrene (PS), polyvinyl chloride (PVC), polylactic acid (PLA) or polyethylene terephthalate (PET). As used herein and unless otherwise stated, “polyethylene” encompasses high density polyethylene (HDPE), low density polyethylene (LDPE), linear low density polyethylene (LLDPE), and ultra low density polyethylene (ULDPE). As used herein and unless otherwise stated, “polypropylene” encompasses homopolymer polypropylene, random copolymer polypropylene, and block copolymer polypropylene. Preferably, all components of the cassette are made of sustainable materials selected from the group consisting of renewable materials, recycled materials, regrind materials, and mixtures thereof, typically wherein “renewable materials” include bio-polyethylene, bio-polyethylene terephthalate, and bio-polypropylene.

In an embodiment, the cassette 1 comprises substantially all wetting components for the filtration unit. Advantages of this arrangement includes ability of easily and quickly retaining and replacing erodible and/or congestable components in a single step.

In an embodiment, the cassette 1 further comprises a third reservoir 11 in fluid communication with the filter 3 and arranged to receive an amount of cell-free solution, preferably wherein the third reservoir 11 is in fluid communication with a sample outlet 6 when connected to a filtration unit 19. Advantages of this arrangement include the possibility of collecting a sample of cell-free solution as the cell solution is filtered during the back and forth flow through the filter, prior to releasing a predetermined amount of cell-free sample solution.

In an embodiment, the third reservoir 11 may be arranged to couple to a third actuator 23 when the cassette 1 is connected to the filtration unit 19. In this embodiment, the third actuator 23 may be arranged to impart an upwardly force substantially simultaneously to the actuation of the first and/or second actuators 8, 9 such to create a suction force that aids flow of cell-free solution through the filter wall 14 and into the third reservoir 11. An advantage of this embodiment is that sufficient pressure is generated to force cell-free solution through the filter wall 14 without having to apply excessive forces by the first and/or second actuators 8, 9. Such ensures effective filtration without excessive pressure build-ups in the system, reducing the risk of leakage and/or failure.

In an embodiment, the cassette 1 further comprises one or more conduits 12, the cassette 1 being arranged such that, preferably only, when connected to the filtration unit 19, said one or more conduits 12 engage with one or more valves 10 to provide a closable connection capable of regulating the flow through said one or more conduits 12 at least, preferably only, between an open and closed position. Typically the conduits 12 are made of a flexible material that is capable of being pinched by the pinch valves in order to block (or stop) flow through a respective portion of said conduits 12, typically the said conduits being made of a material comprising plastics and the like.

In an embodiment, the one or more valves (10) comprise, preferably consist of, one or more pinch valves, preferably comprising a pneumatic cylinder for operating (i.e. activating and de-activating the valve for the flow/no flow conditions). Advantages of this arrangement include effective on/off flow control with highly limited risk of leakage when in the closed position, as well as low cost, crevice free flow path, and the ability to easily change out.

In an embodiment, when connected to the filtration unit 19, the filter 3 is in fluid communication with an air inlet 13 arranged to allow a flow of air through at least said filter to purge any residue left therein following filtering, preferably wherein the air is forced through a fiber wall 14 to a filter outlet 15 and/or through a filter inlet 16 to the filter outlet 15.

In one embodiment, the filter 3 comprises, preferably consists of, a hollow fibrous material comprising a fiber wall 14 projecting from a filter inlet 16 to a filter outlet 15, preferably arranged such to permit a flow of cell-containing solution through said filter inlet and/or outlet 16,15 and a flow of cell-free solution through said fiber wall 14.

In one embodiment, the connector 7 is arranged to adjoin the filtration unit 19 by a, preferably single, transverse movement of the connector 7 with respect to the filtration unit 19 following an insertion axis in a direction substantially perpendicular or parallel to a centerline 17 of the filter 3, preferably wherein said transverse movement comprises sliding of the connector 7 in a receiving portion of the filtration unit 19. Advantages of this arrangement allow for single handed connection and removal of the cartridge whilst appropriately ensuring that the correct predetermined portions of respective conduits engage with respective valves, preferably pinch valves, to avoid incorrect assembly of the components. The connector 7 may comprise a connection joint (not shown) shaped such to fit into the receiving portion of the filtration unit only in when in the suitable orientation. The latter aids to prevent incorrect mounting of the cassette. Locating pins may further be present in the filtration unit to fit into locating holes in the cassette.

In an embodiment, the cassette is arranged such that when it is connected to the filtration unit 19, the filter 3 is in fluid communication with one or more flush solution inlets 18 arranged to allow a flow of flush solution through at least said filter to flush any residue left therein following filtering, preferably wherein the flush solution is forced through a fiber wall 14 to a filter outlet 15 and/or through a filter inlet 16 to the filter outlet 15, and typically wherein the flush solution is selected from the group consisting of water, buffers such as phosphate buffers, caustic solutions such as sodium hydroxide (NaOH), and mixtures thereof, and the like.

The Filtration Unit

The filtration unit generally comprises all moving parts required for triggering the desired flow of solution for filtering, including actuators and valves, and is arranged to receive a cassette as described herein.

The filtration unit comprises the first and second actuators 8,9 and the one or more valves 10, typically housed by a housing 20 arranged to retain said actuators 8,9 and valves 10 in a predetermined position such that when the cassette 1 is connected to the filtration unit, each said valve 10 cooperates with each predetermined position of the conduit(s) of the cassette 1, and each first and second actuator 8,9 cooperates with each first and second reservoir 2,4.

In a preferred embodiment, the filtration unit 19 further comprises a third actuator 23 arranged to couple to the third reservoir 11 when the cassette 1 is connected to the filtration unit 19. In this embodiment, the third actuator 23 may be arranged to impart an upwardly force substantially simultaneously to the actuation of the first and/or second actuators 8, 9 such to create a suction force that aids flow of cell-free solution through the filter wall 14 and into the third reservoir 11. In this arrangement it is preferred that said suction force is less than the first and second forces applied by the first and second actuators 8, 9.

In an embodiment, the first actuator 8 is arranged to apply a first force onto the cell-containing solution in the first reservoir and, substantially simultaneously, the second actuator 9 is arranged to apply a second force onto the cell-containing solution in the second reservoir, wherein the first force is greater than the second force when imparting flow in the first direction, and wherein the first force is less than the second force when imparting flow in the second direction (typically wherein both the first and second forces are in a downwardly direction), preferably wherein both first and second forces are greater than ON.

In a preferred embodiment, the actuators (e.g. each actuator) comprise a plunger preferably sized such to be slidable and leak-tight within the respective reservoirs such to provide an actuation resembling a piston and cylinder arrangement (e.g. the plunger being the piston and the reservoir being the cylinder). It is understood that the plunger may further comprise a mechanical seal common in the art to further reduce the risk of leakage during actuation. The actuators (e.g. each actuator) herein may further comprise a drive that powers the strokes of the plunger, the drive is typically selected from a pneumatic motor or stepper motor, preferably a stepper motor. An advantage of selecting a stepper motor over a pneumatic motor is that the former has been found to work substantially better at low flow rates and pressures. This further enables to minimize membrane fouling and achieve maximum transfer of proteins through the filter wall 14 with the cell-free composition.

In a preferred embodiment, the first actuator 8 and the second actuator 9 are arranged to provide different rates of displacement to respective plungers, wherein one of the two displacements is negative (i.e. in the opposite direction) such that the difference between the flow rate applied to the cell-containing composition by the first actuator 8 and the flow rate applied to the cell-containing composition entering the second reservoir 4 is equal to the flow rate of cell-free solution through the fiber wall 14. An advantage of this arrangement is that the proportion of cell-free composition permeate produced is precisely controlled by accurately controlling the rate of incoming flow and the slower rate into the second reservoir. Particularly when utilizing a stepper motor to achieve such actuation, frictional effects of the plunger (as well as ones typically arising in pneumatic cylinders) are negated, making the operation of controlling flow through the fiber wall 14 very accurate and reliable. This in turn allows for minimizing of membrane fouling and maximizing target protein passage through the fiber wall, as well as increasing the life of the filter and ease of subsequent cleaning of the filter.

In an embodiment the filtration unit is connectable to a bioreactor 21 from which the cell solution is fed from through the sample inlet 5.

In an embodiment, the filtration unit is connectable to a flush solution source (typically including a flush solution reservoir) from which the flush solution is fed from through the flush solution inlet 18.

In an embodiment, the filtration unit is connectable to an air source (typically comprising a compressor or pump) from which the air is fed from through the air inlet 13.

It is further understood that the features of elements retained in the filtration unit (such as valves, and actuators) described in connection with the cassette in the above section are also applicable to the disclosure relating to the filtration unit.

The Process

The process of filtering a cell-containing solution comprises the steps of: optionally connecting a cassette 1 described herein to a filtration unit 19 described herein; providing a filtration unit 19 as described herein comprising a cassette as described herein; allowing an amount of cell-containing solution to flow through the solution inlet 5 and into the first reservoir 2; actuating the first actuator 8 to push the cell-containing solution through the filter 3 in a first direction; actuating a second actuator 9 to push the cell-containing solution through the filter 3 in a second direction; collecting a sample of cell-free solution filtered by said filter 3; optionally removing said cassette 1 from said filtration unit 19 for disposal thereof; wherein said first direction is opposite said second direction.

Preferably in the embodiments herein, the cell-containing composition is allowed (or arranged) to flow through the filter in a continuous back-and-forth in the first and second directions until the filtration of the cell-containing composition is complete.

In an embodiment, the process comprises the further steps of applying a first force by the first actuator 8 and a second force by the second actuator 9 onto the cell containing solution stored in the first and second reservoirs 2,4 respectively, and said first force is greater than said second force when imparting flow in the first direction, and wherein said first force is less than said second force when imparting flow in the second direction, preferably wherein both first and second forces are greater than ON.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure, there is thus no intention of excluding equivalents of the features shown and described or portions thereof, it being recognized that the scope of the disclosure is defined and limited only by the claims which follow.

1. A cassette comprising:
a first reservoir arranged to couple to a first actuator for imparting flow to a cell-containing solution in a first direction through a filter;
a second reservoir arranged to couple to a second actuator for imparting flow to a cell-containing solution in a second direction through the filter;
the filter being in fluid communication with the first and second reservoirs, and when connected to a filtration unit is in further fluid communication to a sample inlet, and a sample outlet;
wherein, the cassette is disposable and housed by a connector configured to be removably coupled to the filtration unit, the filtration unit comprising the first and second actuators and one or more valves.

2. The cassette according to claim 1, wherein the first and second directions are opposite directions.

3. The cassette according to claim 1, wherein the cassette is free of moving parts.

4. The cassette according to claim 1, wherein the cassette comprises substantially all wetting components for the filtration unit.

5. The cassette according to claim 1, further comprising a third reservoir in fluid communication with the filter and arranged to receive an amount of cell-free solution, wherein the third reservoir is in fluid communication with a sample outlet when connected to the filtration unit, wherein the third reservoir is arranged to couple with a third actuator for imparting a suction force substantially simultaneously to the actuation of the first and/or second actuators.

6. The cassette according to claim 1, further comprising one or more conduits, the cassette being arranged such that, when connected to the filtration unit, the one or more conduits engage with one or more valves to provide a closable connection capable of regulating the flow through the one or more conduits between an open and closed position.

7. The cassette according to claim 6, wherein the one or more valves comprise one or more pinch valves.

8. The cassette according to claim 1, wherein, when connected to the filtration unit, the filter is in fluid communication with an air inlet arranged to allow a flow of air through at least the filter to purge any residue left therein following filtering.

9. The cassette according to claim 1, wherein the filter comprises a hollow fibrous material comprising a fiber wall projecting from a filter inlet to a filter outlet, the filter being configured permit a flow of cell-containing solution through the filter inlet and/or outlet and a flow of cell-free solution through the fiber wall.

10. The cassette according to claim 1, wherein the connector is arranged to be coupled to the filtration unit by a transverse movement of the connector with respect to the filtration unit following an insertion axis in a direction substantially perpendicular or parallel to a centerline of the filter.

11. The cassette according to claim 1, wherein, when connected to the filtration unit, the filter is in fluid communication with one or more flush solution inlets arranged to allow a flow of flush solution through at least the filter to flush any residue left therein following filtering.

12. A filtration unit comprising the cassette according to claim 1.

13. The filtration unit according to claim 12 wherein the first actuator is arranged to apply a first force onto the cell-containing solution in the first reservoir and, substantially simultaneously, the second actuator is arranged to apply a second force onto the cell-containing solution in the second reservoir, wherein the first force is greater than the second force when imparting flow in the first direction, and wherein the first force is less than the second force when imparting flow in the second direction.

14. The filtration unit according to claim 12 wherein the first and second actuators comprise a drive and a plunger, the plunger being arranged to reciprocate within the respective first and second reservoirs, and wherein the drive of the first actuator is arranged to provide a first rate of displacement and the drive of the second actuator is arranged to provide a second rate of displacement wherein the first and second displacements are in opposite directions and of a different magnitude adjusted such that friction between the plungers and reservoirs are compensated.

15. A method of filtering a cell-containing solution comprising the steps of:
connecting the cassette according to claim 1 to a filtration unit according to claim 12;
allowing an amount of cell-containing solution to flow through the solution inlet and into the first reservoir;
actuating the first actuator to push the cell-containing solution through the filter in a first direction;
actuating a second actuator to push the cell-containing solution through the filter in a second direction;
collecting a sample of cell-free solution filtered by filter;
removing the cassette from the filtration unit (19) for disposal thereof;
wherein the first direction is opposite the second direction.

16. The method according to claim 15, wherein a first force is applied by the first actuator and a second force is applied by the second actuator, and the first force is greater than the second force when imparting flow in the first direction, and wherein the first force is less than the second force when imparting flow in the second direction.

17. A method according to claim 15, wherein the first and second actuators comprise a drive and a plunger, the plunger being arranged to reciprocate within the respective first and second reservoirs, and wherein the drive of the first actuator is arranged to provide a first rate of displacement and the drive of the second actuator is arranged to provide a second rate of displacement and wherein the first and second displacements are in opposite directions and of a different magnitude adjusted such that friction between the plungers and reservoirs are compensated, wherein the difference between the flow rate of the cell-containing composition out of the first reservoir and the flow rate of said cell-containing composition into the second reservoir is equal to the flow rate of cell-free solution through the fiber wall of the filter.

18. The filtration unit according to claim 14, wherein the difference between the flow rate of the cell-containing composition out of the first reservoir and the flow rate of the cell-containing composition into the second reservoir is equal to the flow rate of cell-free solution through the fiber wall of the filter.

19. The filtration unit according to claim 18, wherein the drive of the first actuator comprises a first stepper motor and the drive of the second actuator comprises a second stepper motor.

20. A method of filtering a cell-containing solution comprising the steps of:
connecting the cassette according to claim 5 to a filtration unit according to claim 13;
allowing an amount of cell-containing solution to flow through the solution inlet and into the first reservoir;
actuating the first actuator to push the cell-containing solution through the filter in a first direction;
actuating a second actuator to push the cell-containing solution through the filter in a second direction;
actuating the third actuator substantially simultaneously to the first and/or second actuator to provide a suction force aiding flow of cell-free solution through the filter wall and into the third reservoir;
collecting a sample of cell-free solution filtered by the filter;
removing the cassette from the filtration unit for disposal thereof,
wherein the first direction is opposite the second direction.
